(19) United States
(12) Patent Application Publication
(10) Pub. No.: US 2022/0037009 A1
(43) Pub. Date: Feb. 3, 2022

(54) Health and Lifestyle Exploration Based User Engagement Application

(71) Applicant: LifeQ B.V., Amsterdam (NL)

(72) Inventors: Laurence Olivier, Alpharetta, GA (US); Riaan Conradie, Amsterdam (NL)

(21) Appl. No.: 17/277,097

(22) PCT Filed: Sep. 23, 2019
(86) PCT No.: PCT/US2019/052372

§ 371 Date: Mar. 17, 2021

Related U.S. Application Data
(60) Provisional application No. 62/734,858, filed on Sep. 21, 2018.

(51) Int. Cl. G16H 40/63; G16H 20/70

(52) U.S. Cl. CPC G16H 40/63; G16H 10/20; G16H 20/70

(57) ABSTRACT

A system and method for a user to select and implement a lifestyle intervention. The system and method allows the user to select a lifestyle intervention and define the parameters of the intervention. The system monitors physiological parameters of the user that are related to the lifestyle intervention.

### REFERENCE TO RELATED APPLICATION

This application claims priority to U.S. Provisional Application No. 62/734,858, filed on Sep. 21, 2018, which is relied upon and incorporated in its entirety by reference.

### FIELD

This disclosure is generally related to health devices and, more specifically, to systems, methods and devices for providing health and lifestyle intervention options in combination with other users and/or context information via a communications network.

### BACKGROUND

The number of exercise devices and software applications available to users has grown exponentially over the last decade. These devices and software applications can encourage their users to carry out tasks, including physical activities (e.g., get up and walk), intake food and liquids (e.g., drink water), or other activities (e.g., go to bed). These applications and devices utilize “tips and nudges” in an attempt to influence user behavior. The forms of participation around the engagement that the user and the business play is as follows. In a “tips and nudges” regime, the application, or application provider, plays an active and invasive role, i.e. a parental, “big brother” approach and attempts to forcefully change behavior.

On the other hand, the application user plays a passive role. Their behavior is defined, and limited, by the scope of the “tips and nudges” they receive, and the choices they make to act on or ignore these “tips and nudges”. That is, the user has no say in the types of tips and nudges that are provided.

In addition, people are being overwhelmed with conflicting and ever- changing advice and tips related to health and lifestyle recommendations. Users find it extremely difficult to know which lifestyle interventions and health tips would be most beneficial their unique body and easiest to follow and sustain. Therefore, there is a need for systems and methods that allow individuals a chance to participate in the types of options received, as well as to navigate the numerous options available.

### SUMMARY OF THE INVENTION

Embodiments of the present invention are directed to Health and Lifestyle Explorer (HLE) systems, methods and devices that facilitate a user's understanding of how lifestyle changes affect their unique body. Individuals that have a deep interest in their health may find conventional wearable devices and user engagement software applications overwhelming in terms of engagement and underwhelming in terms of novel learnings and useful feedback.

The HLE provides lifestyle interventions and health tips that are determined to be most beneficial to the user's own attributes and subjective perspective—those directed to the user's unique body and sustainability. In some embodiments, the HLE may compliment and facilitate the user's interaction with other systems, devices, software modules, etc., such as third-party software applications and wearable devices.

In an aspect, the invention is directed at a method of electronically assisting a user with a lifestyle intervention that includes providing the user with a plurality of lifestyle interventions, receiving a selection of one of the plurality of lifestyle interventions, acquiring physiological parameters of the user associated with the selected lifestyle intervention, and providing to the user the acquired physiological parameters. In some instances, providing to the user the acquired physiological parameters can also include providing current physiological parameters. In some instances, the initial physiological parameters can include physiological parameters of the user when the selection of one of the plurality of lifestyle interventions was received. The method can provide both current and initial physiological parameters, both or either of which can be provided continuously throughout the intervention. In some aspects, the user can terminate the lifestyle duration before its completion. In another aspect, the method of selecting a lifestyle intervention can include providing a feedback survey upon termination of the lifestyle intervention. In an aspect, the selection of a lifestyle intervention can include parameters selected by the user relating to the selected lifestyle intervention. The selected parameters can include a duration of the lifestyle intervention, as well as types of notices. In other aspects, the parameters and lifestyle interventions can be based upon interventions and parameters collected from community members of the HLE.

In another aspect, the invention is directed to a health and lifestyle exploration system for use by a user, where the system includes at least one computing device with a processor, memory, a transmitter and a user interface. In such an aspect, the processor is configured to provide to the user at least one lifestyle intervention, receive a user-selected lifestyle intervention, acquire physiological parameters of the user associated with the selected lifestyle intervention, and provide to the user the acquired physiological parameters. In an aspect, the user interface includes a graphical user interface. In an aspect, the system can be configured to present a plurality of lifestyle interventions. These plurality of lifestyle interventions can be generated by community members of the HLE, or from the user themselves.

In an aspect, the system can be configured to provide current physiological parameters to the user, as well as the initial physiological parameters which are collected when the processor receives the user-selected lifestyle intervention. These physiological parameters can be provided continuously throughout the intervention. In an aspect, the system includes a wearable device having a plurality of sensors to acquire data steams relating to the acquired physiological parameters, wherein the wearable device, which is in communication with other components of the system.

In an aspect, the use-selected lifestyle intervention can include parameters selected by the user relating to the user-selected lifestyle intervention. The selected parameters can include a duration of the lifestyle intervention. In an aspect, the system can provide a feedback survey upon termination of the lifestyle intervention. In addition, the system allows the user to terminate the lifestyle intervention before the duration is completed.

These and other objects and advantages of the invention will become apparent from the following detailed description of the preferred embodiment of the invention.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### BRIEF DESCRIPTION OF THE DRAWINGS

The accompanying drawings, which are incorporated herein and form part of the specification, illustrate the present disclosure and, together with the description, further serve to explain the principles of the disclosure and to enable a person skilled in the relevant art to make and use the disclosure.

FIG. 1 illustrates an exemplary graphical user interface of a health and lifestyle explorer in a first mode according to an aspect of the present invention.

FIG. 2 illustrates an exemplary graphical user interface of a health and lifestyle explorer in a second mode according to an aspect of the present invention.

FIG. 3 illustrates an exemplary graphical user interface of a health and lifestyle explorer in a third mode according to an aspect of the present invention.

FIG. 4 illustrates an exemplary graphical user interface of a health and lifestyle explorer in a fourth mode according to an aspect of the present invention.

FIG. 5 illustrates an exemplary graphical user interface of a health and lifestyle explorer in a fifth mode according to an aspect of the present invention.

FIG. 6 illustrates an exemplary graphical user interface of a health and lifestyle explorer in a sixth mode according to an aspect of the present invention.

FIG. 7 illustrates an exemplary graphical user interface of a health and lifestyle explorer in a seventh mode according to an aspect of the present invention.

FIG. 8 illustrates an exemplary graphical user interface of a health and lifestyle explorer in an eighth mode according to an aspect of the present invention.

FIG. 9 illustrates an exemplary graphical user interface of a health and lifestyle explorer in a ninth mode according to an aspect of the present invention.

FIG. 10 illustrates a schematic representation of a computing device according to an aspect of the present invention.

FIG. 11 illustrates a schematic representation of a Health and Lifestyle Explorer system according to an aspect of the present invention.

### DETAILED DESCRIPTION

Although specific configurations and arrangements may be discussed, it should be understood that this is done for illustrative purposes only. A person skilled in the pertinent art will recognize that other configurations and arrangements can be used without departing from the spirit and scope of the present invention. It will be apparent to a person skilled in the pertinent art that this invention can also be employed in a variety of other applications beyond those specifically mentioned herein. It should be appreciated that the particular implementations shown and described herein are examples and are not intended to otherwise limit the scope of the application in any way.

It is noted that references in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of one skilled in the art to effect such feature, structure or characteristic in connection with other embodiments whether or not explicitly described.

### Overview

The Health and Lifestyle Explorer (HLE) systems, methods and devices disclosed herein provide users with the ability to select from a wide and rich set of opportunities and tools that the user may choose to leverage in a process of discovery and self-improvement. The user is enabled to play an active role in choosing the time, opportunity, area of focus for improvement, scope, duration, and relevant tools in order to effect a desired outcome. Thus, the HLE approach is simplified to focus on the user as the active driver.

In an aspect, the HLE system utilizes various computing devices having user interface(s) with which the user can interact. In exemplary aspects, the HLE systems can utilize graphical user interfaces that are associated with various computing devices (e.g., smart phones, smart watches/monitors, personal computers, etc.).

In addition, the HLE systems can utilize various computing devices that acquire, via themselves or other devices, biological metrics/physiological information for their users. The devices can include a variety of sensors, including, but not limited to, PPG, temperature, motion (e.g., gyroscope, accelerometer, inertial measurement units), electrodes (EEG, EKG, EMG), and the like. The sensors collect data streams that can then be later used to determine various physiological characteristics of the user, including, but not limited to, heart rate, exercise rate, oxygen consumption, lung capacity, stress levels, sleep quality, weight, exercise quality rate, caloric rate, and various other known biological metrics known in the art. In an aspect, the computing devices utilized to generate the biological metrics can include, but not limited to, those disclosed in U.S. application Ser. Nos. 14/128,675; 15/118,183; 15/521,667; 15/123,082; 15/521,667; 15/330,865; 15/343,950; 15/393,398; 15/393,982; 15/393,398; 15/415,443; 15/821,206; 15/721,079; 16/190,080; and 16/058,668, all of which are incorporated by reference in their entirety herein.

This HLE approach has the advantage over previous approaches as it has eliminated elaborate or long-winded processes required to train or converge complex models and/or outcomes. This results in very short return on investment (ROI) times. Additionally, due to the simplicity of the approach and the focus on the user as the active participant, the technology and resources required to implement, enhance, and maintain the solution are considerably simpler and lighter than for the traditional lifestyle intervention “tips & nudges” approach.

The HLE systems, methods, and devices described herein passively provide insights to an HLE user regarding potentially effective lifestyle and health changes. In other words, instead of an application providing interruptions or notices automatically, the HLE systems of the present invention present the lifestyle interventions (notices/interruptions) at the client's demand. In an aspect, the HLE user is enabled to determine which interventions are of current interest and is then provided an engine to pursue and assess the effectiveness of a given intervention. For example, if an HLE user frequently engages in excess screen time, and wants to decrease screen time, the HLE user can pursue an intervention to reduce screen time for a month, and assess the effectiveness and ease of the intervention.

In some embodiments, the intuitive HLE system assists the HLE user in learning how any lifestyle change affects their own unique attributes by tracking and monitoring various biological metrics that are associated with the requested changes. The HLE user is enabled to understand what lifestyle changes are the most sustainable and beneficial to their unique body through information driven self-exploration/experimentation. By allowing the user to determine the lifestyle change, tracking the progress, and obtaining feedback directly from the user, the HLE is active in pursuing the lifestyle changes and seeing the impacts in real time.

The embodiments provide a new type of functionality (e.g., providing the user the opportunity to select the lifestyle change and ways to impact it) that is not available in other applications and take an active role to communicate this differentiation to its users. The embodiments can be utilized for real world interventions (e.g., medicine, improved habit-forming, etc.) as well as for digital interventions (e.g., self-help software applications, health programs, etc.) These and other aspects are discussed below.

Embodiments described herein can additionally integrate with other user engagement or wellness software applications. For example, embodiments can be configured to integrate with other modules, including third party applications, that perform data syncing (in embodiments, this functionality can remain in the standard wearable device app). In other aspects, the HLE system can utilize the feedback and results of all users to maintain and update the interventions and parameters associated with them.

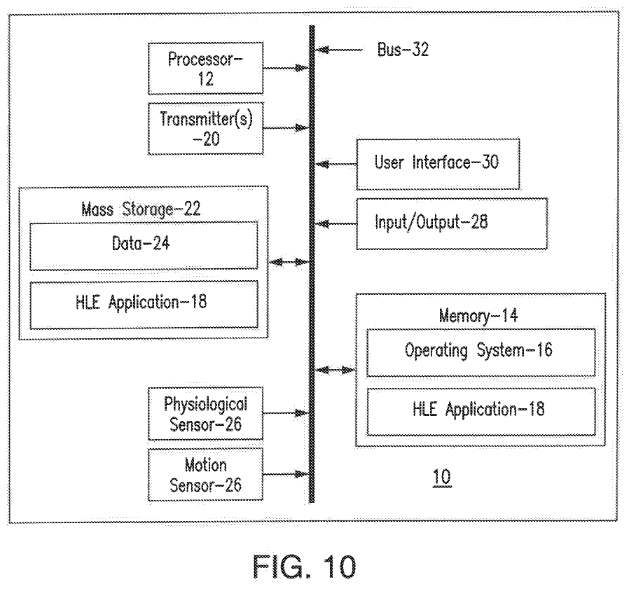

In an aspect, the HLE system can include various computing devices. FIG. 10 illustrates an embodiment of a computing device 10 that can be utilized by the system. As shown, the computing device 10 includes a processor 12, memory 14, operating systems 16, an HLE application 18, various wireless transmitters 20 (e.g., WiFi, Bluetooth, cellular, NFC, GPS, etc.), mass storage 22 for storing data 24 and applications 18, various sensors 26 for monitoring physiological characteristics of the user and their activity as discussed above, input/outputs 28, and various user interfaces 30, all connected by a bus 32. The user interfaces 30, can include, but are not limited to, displays, touch-screen displays, microphone/speakers, and the like. In a preferred embodiment, at least one of the computing devices 10 is configured to interact with the user via a graphic user interface 30 on a touch-screen display that allows user engagement with the systems and programs of the HLE system.

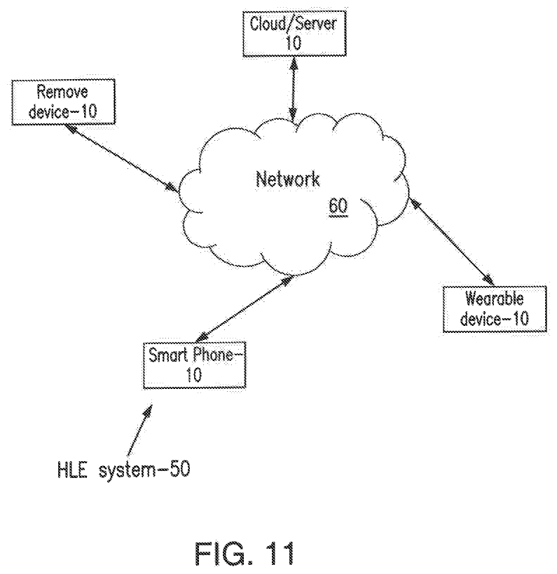

As discussed above, various computing devices 10 can be utilized by the HLE system 50. FIG. 11 illustrates the HLE system 50 according to an aspect of the present invention. The system 50 can utilize wearable devices 10, smart phones 10, remote devices 10 that can transmit other data (e.g., weight scale, exercise equipment, etc.), and remote servers 10, all of which are in communication with one another over various networks 60. In combination, the HLE system 50 needs to be able to collect and calculate biological metrics of the user as well as provide means for the user to choose and monitor a lifestyle intervention.

Objects of the present embodiments include providing HLE users with a periodic health snapshot, which includes biological metrics of the user, as a form of “light touch” user engagement distinct from the conventional “tips and nudges” engagement approach. Thereby, HLE users can engage by receiving personal learning and insights that do not flow from conventional approaches. HLE users are provided with the ability to decide for themselves what lifestyle intervention they would like to try, and for how long they would like to try the intervention. HLE users can track all the lifestyle interventions they have tried and log of how effective these interventions were. HLE users are enabled to compare their before and after physiological snapshots (health snapshots or “selfies”) to visualize what lifestyle interventions were the most effective.

Thus, the HLE systems, methods, and devices facilitate informing the HLE user as to what interventions are both sustainable and most beneficial to their health, in a manner that is not overwhelming or disengaging. The user is rewarded with novel learnings of their own lifestyle and how it affects their unique physiology.

### Embodiments of a Health and Lifestyle Explorer Device

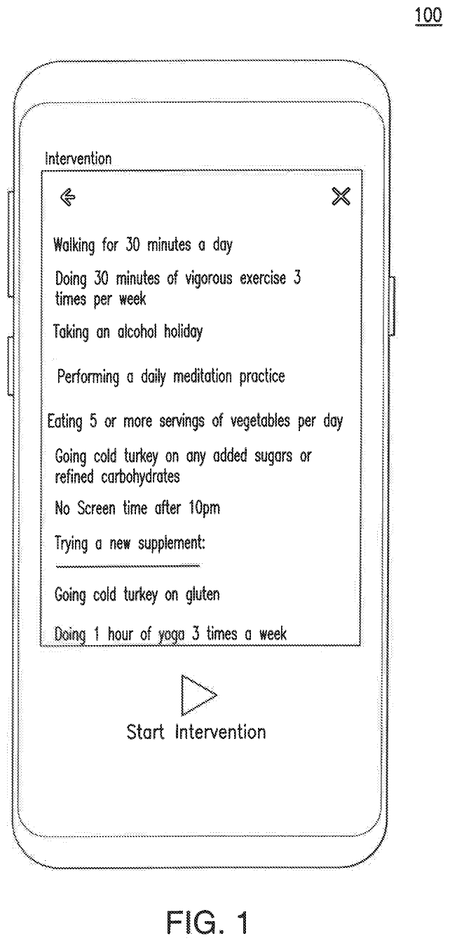

According to an aspect of the HLE system, as illustrated in FIGS. 1-9, a user is provided a simplified graphical user interface that enables the user to select a lifestyle intervention. In an aspect, the user is able to select from a wide and rich set of opportunities and tools that the user may choose to leverage in a process of discovery and self-improvement. The HLE system provides users with a feed of health information to give them ideas of what life interventions they can try out. Over time, this information may become more targeted using information streams that reflect physiological parameters associated with the user.

In an aspect, the HLE system can provide graphical user interface (GUI) in a first mode, such as illustrated in FIG. 1. In the first mode, the GUI 100 provides a set of potential health and lifestyle interventions that an HLE user may select to engage in. In an example, in the first mode 100, the GUI may provide a set of popular lifestyle intervention options. These options include, but are not limited to, walking for 30 minutes a day, doing 40 minutes of vigorous exercise 3 times a week, taking an alcohol holiday, performing a daily mediation practice, eating 5 or more servings of vegetables per day, going cold turkey on any added sugars or refined carbohydrates, no screen time after 10 om, trying a new suppliant, going cold turkey on gluten, doing 2 hour of yoga 3 times a week, etc. Various other lifestyle interventions can be provided to the user. In an aspect, the lifestyle interventions can be preloaded to the system. In other aspects, the HLE system can allow for the user to add and build the lifestyle interventions, or pull from lifestyle interventions created/collected by other users of the HLE system.

Additionally or alternatively, the particular set of lifestyle options provided in the set may be defined, refined, highlighted or otherwise made available based on the HLE user's connections. For example, the HLE user may connect to friends, family, associates, etc., through the HLE GUI or through other third-party software applications. The suggested interventions set forth may be defined, for example, in the GUI. Thus, the GUI may provide an opportunity to allow multiple HLE users (e.g., colleagues, families, spouses, friends and others who may be socially connected) to start the same intervention for the same duration. The GUI may suggest, for example: “Hey, your friend Sarah just started intervention x that will last for y time. Do you want to join this lifestyle exploration with her?”. Furthermore, the HLE can allow users to specify their own intervention goal regardless of whether the intended goal is contained in a pre-populated set. An HLE user selects a particular goal that they may have. Upon selection of goal the HLE commences tracking data associated with the goal, discussed in detail below. At a future date, the HLE user can measure progress against their selected goal, in a user-driven engagement approach.

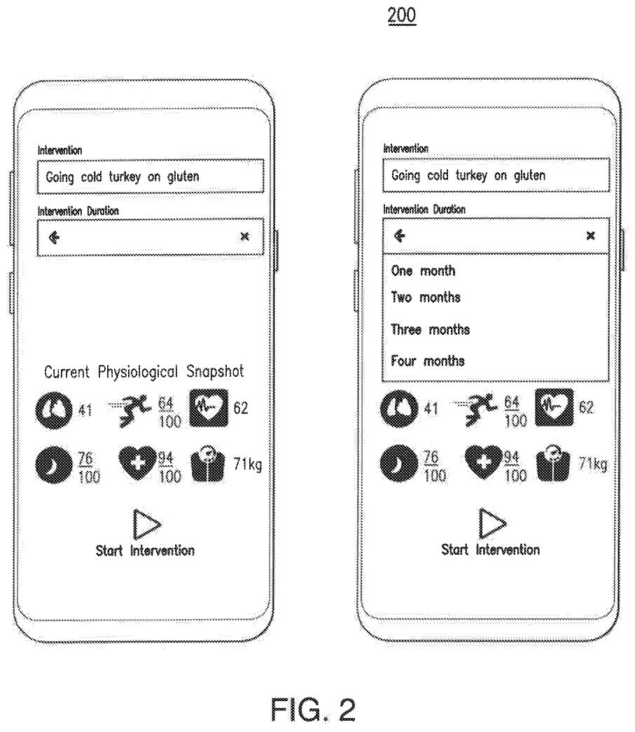

A second exemplary mode 200 of the GUI is illustrated in FIG. 2. As shown, the HLE user may define the parameters of the intended intervention. For example, as shown in FIG. 2, after the user has selected choosing going cold turkey on gluten, the user can select the duration of the intervention (e.g., 1-4 months). In some embodiments, the HLE user may be restricted to pursuing one intervention at a time, to facilitate the HLE user's motivation to complete the goal and to more clearly identify whether a specific goal works for a user.

For example, the HLE user may select a duration period for the intervention. In the HLE approach, the intended goal is not “losing weight,” “reduce heart rate,” or the like. Instead, the HLE user is enabled to actuate their own lifestyle change. Subsequently, the HLE user will be engaged by comprehensive health data, i.e., a health snapshot, associated to the HLE user. Thereby, the HLE user can visualize the effectiveness of the selected intervention. In an aspect, the snapshot can include information related but not limited to lung capacity, an overall health score, blood pressure, heart rate, sleep scores, weight, and the like.

Instead of selecting an abstract goal, the user can measure an intervention based upon a specific characteristics/physiological parameter. For example, instead of an abstract goal of “improved sleep quality,” the HLE user can select an intervention such as “no screen time two hours before bedtime.” The HLE user can set the intended duration of the intervention and track progress toward the goal. In the future, the HLE user may select a different intervention for experimentation to help out with sleep quality, such as “no exercise four hours before bedtime.” In another example, an HLE user may select an intervention, such as eliminating gluten from their diet for a set period, to see how the intervention can impact their health. Here, the user can pick a specific action item that can impact a specific goal. In addition, if the lifestyle intervention requires a nudge or encouragement, the user can select the types of nudges and/or frequency.

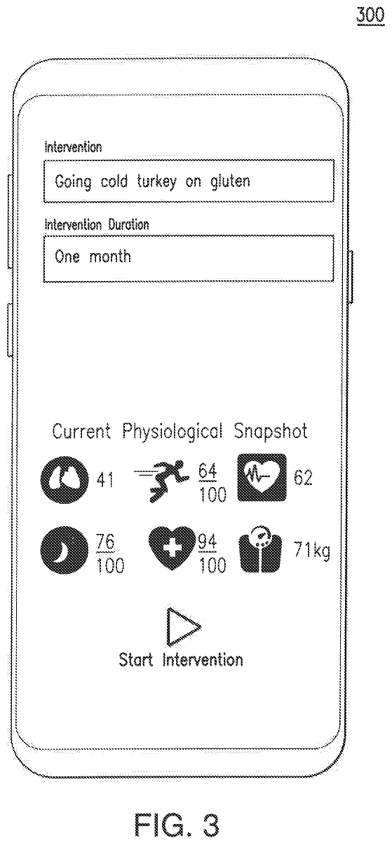

A third exemplary mode 300 of the GUI is illustrated in FIG. 3. As shown, the HLE user may confirm the specific activity and thereby initiate the intended intervention and other parameters, such as duration. The HLE user can view a snapshot of their health at the outset of the intervention. Subsequently, the HLE user can view their snapshot at throughout the duration period of the intervention (not shown in FIG. 3), and compare their snapshot attributes.

Thus, for example, instead of an abstract goal, such as “sleep more per day,” the HLE user selects an intervention to pursue. The HLE can provide a comprehensive score for the quality of sleep. Individuals respond in different ways to different interventions. Thus, for one HLE user, reducing screen time may have a huge impact to sleep quality, while another HLE user may not receive much benefit.

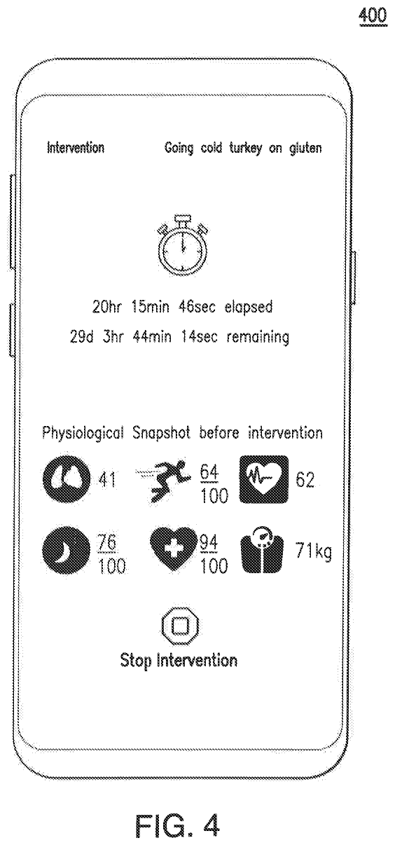

In a fourth exemplary mode 400 of the GUI, illustrated in FIG. 4, an HLE user may view the period of duration completed of the intervention, the remaining time left for the intervention, and their health snapshot at initiation of the intervention. Furthermore, the health snapshot can be recalculated periodically, for example, on a daily basis, to enable the HLE user to visualize their overall progress. Thus the HLE provides a feedback loop that is constantly updated with real time, continuous information. This information may be retrieved from a separate module, such a wearable device or application software. For example, the information may be displayed via the GUI by an HLE application running on an individual's phone, with the biological parameters being collected and calculated by wearable devices and remote servers separately or in combination.

In addition, the user has the ability to make the ultimate determination as to whether or not the selected intervention should be continued or not. At any time, for example based on the feedback, the HLE user may choose to stop the intervention. When the HLE user selects to stop the intervention, the HLE may prompt the user to specify a reason why the intervention was terminated.

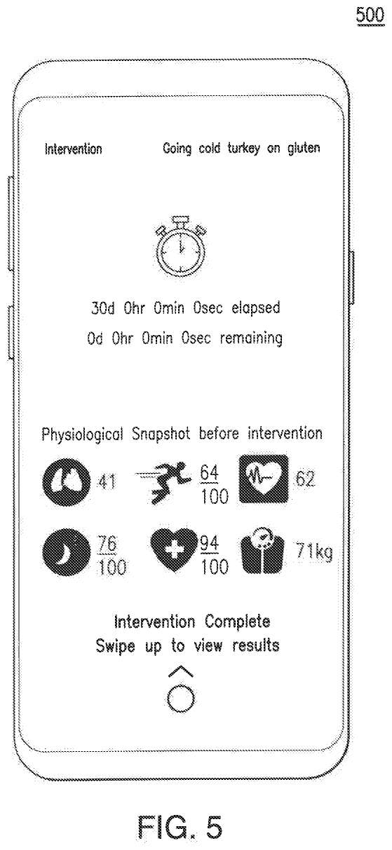

In a fifth exemplary mode 500 of the GUI, illustrated in FIG. 5, an HLE user may be notified of the conclusion of the intervention period. At this point, the HLE user is provided access to their health snapshot as it exists at the time the intervention concludes. The HLE user can thus compare their post-intervention health snapshot with their pre-intervention health snapshot.

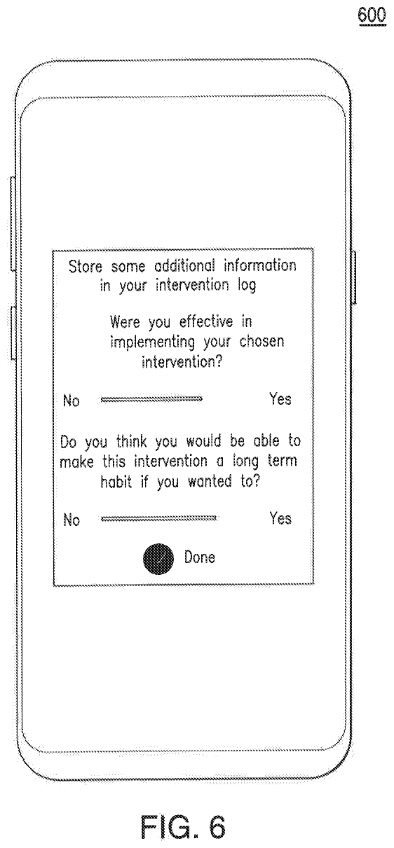

Subsequently, as shown in exemplary mode 600 of FIG. 6, the HLE user can be queried to log information about their intervention experience. For example, the HLE user may provide subjective perceptions about the effectiveness and ease of the intervention that he or she pursued. For example, as discussed with respect to FIG. 4, in some cases the HLE user may choose to terminate a selected intervention prior to completion. In that case, the HLE user may specify reasons for terminating, such as difficulty in pursuing the intervention or lack of perceivable health improvements. Such information about their intervention experience may also be requested at the end of the intervention, so that the HLE system and the HLE user can ascertain how effective this particular intervention was for the user. Such information may be compared to information about other interventions to allow a user to identify a preferred intervention for that particular user. This also provides valuable context to understand what interventions can become habits.

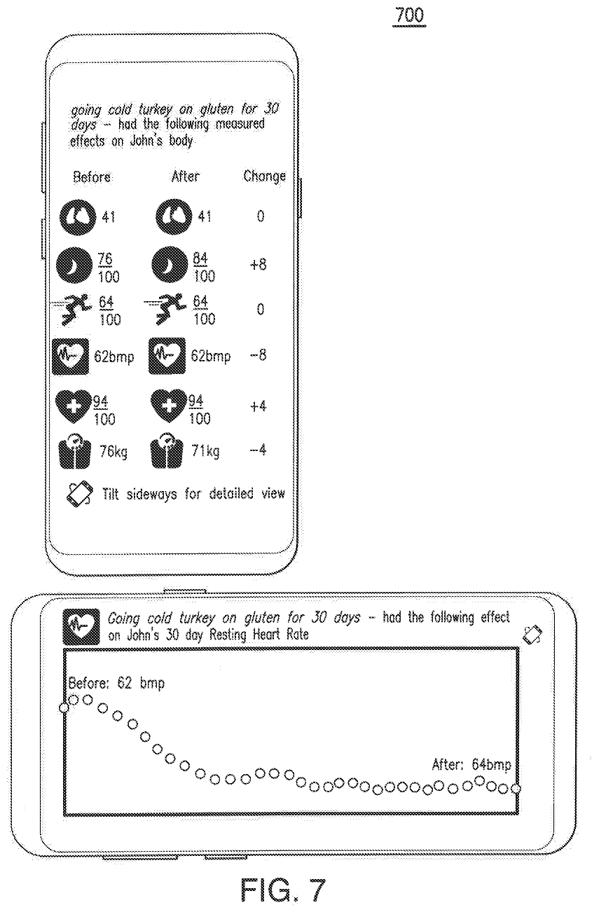

As shown in exemplary mode 700 of FIG. 7, the HLE renders a graphical view of the HLE user's final health snapshot in comparison to their health snapshot at the outset of the intended intervention. For example, after the user completes go cold turkey on gluten for 30 days, the comparison shows that the user's heart rate has decrease from 62 bpm to 54 bpm, while increasing the sleep quality from 76/100 to 84/100. Furthermore, the HLE user can view specific or comprehensive health datum at each recorded interval, such as each daily interval, over the duration of the intervention period. Thus, the HLE user is enabled to visualize the effectiveness of a given intervention over the duration period. This allows users to get a before, after, and change “snapshot” once the desired intervention is concluded, and provide time course data for changes if desired.

In some embodiments, the HLE user can see population averages and benchmarks (e.g., as a percentile) from the global HLE community or user base. Alternatively or additionally, in some embodiments, the HLE user may see their progress in as compared to their personal connections.

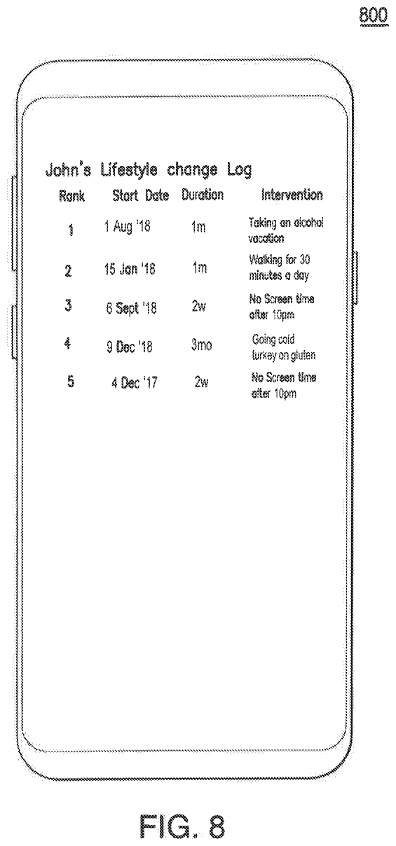

As shown in exemplary mode 800 of FIG. 8, the HLE user may view all past interventions, including each intervention's qualitative rank. The rank of each intervention may be based on how beneficial an intervention is to the user's body (e.g., based on metrics being tracked) or ranked based on how it affects a specific marker (such as heart health or mood).

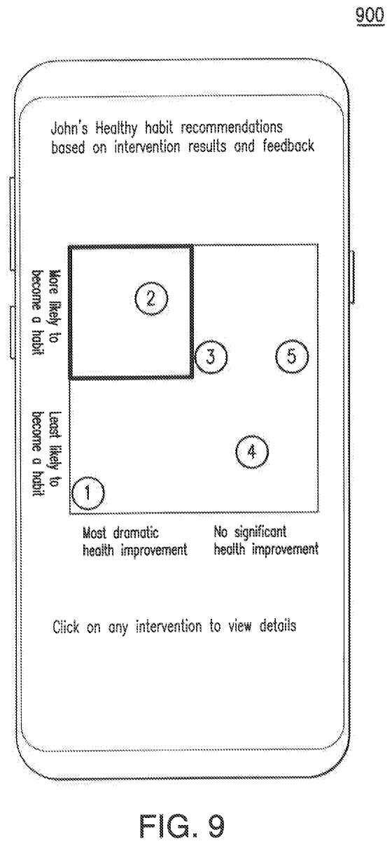

As shown in exemplary mode 900 of FIG. 9, the HLE may provide an intervention visualization. In the intervention visualization, the HLE user can see the benefit vs. ease of each given intervention in relation to other interventions. For example, each intervention can be plotted on a chart, where the vertical axis provides a range of whether an intervention is more or less likely to become a habit, and the horizontal axis provides a range as to whether the health improvement was dramatic or not significant. For example, as shown in FIG. 9, the HLE user can see that intervention “2” was relatively easy to adopt and effective towards health improvement. In contrast, intervention “4” was harder to adopt and less effective. Therefore, the HLE may enable the user to define interventions from past attempts that are suitable to habit forming.

For example, an HLE user may note that the most dramatic health improvements were achieved through intervention “1.” If the HLE user has a particularly high level of personal discipline, the HLE user may select that intervention as suitable to incorporate into their ongoing, daily life. Whereas, an HLE user having less discipline may opt to form pursue a different intervention as an ongoing habit.

Having thus described exemplary embodiments of the present invention, those skilled in the art will appreciate that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A method of electronically assisting a user with a lifestyle intervention, comprising:
a. providing the user electronically with a plurality of lifestyle interventions;
b. receiving a selection of one of the plurality of lifestyle interventions;
c. acquiring physiological parameters of the user associated with the selected lifestyle intervention; and
d. providing to the user the acquired physiological parameters.

2. The method of claim 1, wherein providing to the user the acquired physiological parameters comprises providing current physiological parameters.

3. The method of claim 1, wherein the providing to the user the acquired physiological parameters comprises providing to initial physiological parameters of the user, wherein the initial physiological parameters comprise physiological parameters of the user when the selection of one of the plurality of lifestyle interventions was received.

4. The method of claim 3, further comprising providing current physiological parameters with the initial physiological parameters.

5. The method of claim 1, wherein the receiving the selection of the one of the plurality of lifestyle interventions further comprises;
I. receiving the selection of the lifestyle intervention; and
II. receiving parameters selected by the user relating to the selected lifestyle intervention.

6. The method of claim 5, wherein the selected parameters comprise a duration of the lifestyle intervention.

7. The method of claim 5, further comprising providing a feedback survey upon termination of the lifestyle intervention.

8. The method of claim 7, further comprising receiving a request from the user to terminate the lifestyle intervention before the duration is completed.

9. The method of claim 5, wherein providing to the user the acquired physiological parameters comprises providing current physiological parameters continuously through the duration.

10. The method of claim 5, wherein the selected parameters comprise type of notices.

11. A health and lifestyle exploration system for use by a user, the system comprising:
a. at least one computing device comprising;
I. a processor;
II. memory;
III. transmitters; and
IV. a user interface, wherein the processor is configured to:
A. provide to the user at least one lifestyle intervention;
B. receive a user-selected lifestyle intervention;
C. acquire physiological parameters of the user associated with the selected lifestyle intervention; and
D. provide to the user the acquired physiological parameters.

12. The system of claim 11, wherein the processor provides current physiological parameters to the user.

13. The system of claim 12, wherein the processor further provides initial physiological parameters of the user, wherein the initial physiological parameters are collected when the processor receives the user-selected lifestyle intervention.

14. The system of claim 11, wherein the user-selected lifestyle intervention further comprises parameters selected by the user relating to the user-selected lifestyle intervention.

15. The system of claim 14, wherein the selected parameters comprise a duration of the lifestyle intervention.

16. The system of claim 15, wherein the processor is further configured to provide a feedback survey upon termination of the lifestyle intervention.

17. The system of claim 16, wherein the process is further configured to receive a request from the user to terminate the lifestyle intervention before the duration is completed.

18. The system of claim 15, wherein the processor is further configured to provide current physiological parameters continuously through the duration.

19. The system of claim 11, further comprising a wearable device having a plurality of sensors to acquire data steams relating to the acquired physiological parameters, wherein the wearable device is in communication with the at least on computing device.

20. The system of claim 11, wherein the user interface comprises a graphical user interface.
